(19) United States
(12) Patent Application Publication
(10) Pub. No.: US 2017/0322125 A1
(43) Pub. Date: Nov. 9, 2017

(54) SOLUTIONS FOR SAMPLE PROCESSING

(71) Applicant: RareCyte, Inc., Seattle, WA (US)

(72) Inventor: Daniel Campton, Seattle, WA (US)

(73) Assignee: RareCyte, Inc., Seattle, WA (US)

(21) Appl. No.: 15/492,219

(22) Filed: Apr. 20, 2017

Related U.S. Application Data
(60) Provisional application No. 62/331,331, filed on May 3, 2016.
(60) Provisional application No. 62/367,801, filed on Jul. 28, 2016.

(51) Int. Cl. G01N 1/30; G01N 1/36; C12Q 1/68

(52) U.S. Cl. CPC G01N 1/30; G01N 1/36; C12Q 1/6806; G01N 2001/364; G01N 2001/302

(57) ABSTRACT

This disclosure is directed to solutions and methods of using the solutions. A first solution includes a polymer, a polyol, and a buffer. A second solution includes a polymer, a polyol, and an alcohol.

### CROSS-REFERENCE TO A RELATED APPLICATION

This application claims the benefit(s) of Provisional Application No. 62/331,331, filed May 3, 2016, and of Provisional Application No. 62/367,801, filed Jul. 28, 2016.

### TECHNICAL FIELD

This disclosure relates generally to analyzing a suspension and, in particular, to solutions for covering a sample on a substrate.

### BACKGROUND

Current diagnostic methods and techniques rely on the interpretation of histological and cytological samples. It is an important aspect in these interpretations that the sample is properly imaged for subsequent analysis and processing. Valuable diagnostic information may be obtained from the sample upon subsequent processing due to the quality of the images collected.

### DETAILED DESCRIPTION

This disclosure is directed to solutions and methods of using the solutions. A first solution includes a polymer, a polyol, and an alcohol. A second solution includes a polymer, a polyol, and a buffer.

In the following description, the term “sample” is used to describe a specimen to be analyzed. The specimen may be a suspension, a portion of the suspension, or a component of the suspension. For example, when the suspension is anticoagulated whole blood, the sample may be the anticoagulated whole blood (i.e. a suspension), the buffy coat (i.e. a portion of the suspension), or a cell, such as a circulating tumor cell (i.e. a component of the suspension).

### Solutions

A first solution includes a polymer, a polyol, and a buffer. The polymer, such as polyvinyl alcohol (“PVA”) or polyvinylpyrrolidone (“PVP”), may have a final concentration of approximately 5-20% by mass of the first solution. The polymer may cause the first solution to transition from an aqueous state to a hardened state. However, working in conjunction with the polyol, the polymer and the polyol may keep the first solution in a gelatinous or a -gel-like state. Furthermore, the polymer, such as PVA or PVP, does not immediately (for example, within up to 12 hours) or significantly (for example, reduce emission percentage by more than 50 percent) quench a quantum dot.

The polyol (including, but not limited to, glycerol, propylene glycol, pentaerythritol) may have a final concentration of approximately 5-30% by mass of the first solution. The polyol prevents freezing during storage and has a high refractive index (closer to glass than water).

Components of the buffer may have individual final concentrations of approximately 0.1-100 millimolar. The buffer, which keeps the first solution within a desired pH range, may include, but is not limited to a tris-EDTA buffer (“TE buffer”) or a phosphate buffer. For example, when the TE buffer is used, tris(hydroxymethyl)aminomethane (“tris”) and ethylenediaminetetraacetic acid (“EDTA”) may each have final concentrations of 1-100 millimolar and 0.1-10 millimolar, respectively. When the phosphate buffer is used, sodium phosphate dibasic and/or potassium phosphate monobasic have final concentrations of 0.1-100 millimolar, individually. In one embodiment, the first solution may have the following formulation: 10% glycerol by mass, 10% PVA by mass, 20 mM tris, and 2 mM EDTA.

A second solution includes at least one polymer, a polyol, and an alcohol. The at least one polymer, such as polyvinyl alcohol (“PVA”) or polyvinylpyrrolidone (“PVP”), may have a final concentration of approximately 0.1-5% by volume of the second solution. The polymer may cause the first solution to transition from an aqueous state to a hardened state. However, working in conjunction with the polyol, the polymer and the polyol may keep the first solution in a tacky, a gelatinous, or a gel-like state. Furthermore, the polymer, such as PVA or PVP, does not immediately (for example, within up to 12 hours) or significantly (for example, reduce emission percentage by more than 50 percent) quench a quantum dot. When two polymers, PVA and PVP, are used, each may have a final concentration of approximately 0.1-5% by volume of the second solution.

The polyol (including, but not limited to, glycerol, propylene glycol, pentaerythritol) may have a final concentration of approximately 0.1-5% by volume of the second solution. The polyol prevents freezing during storage and has a high refractive index (closer to glass than water).

The alcohol, such as ethanol and methanol, may have a final concentration of approximately 70-90% by volume of the second solution. The alcohol may accelerate drying and allow for even spreading (for example, the alcohol may lower surface tension and viscosity).

The second solution may also include a buffer, which keeps the first solution within a desired pH range, may include, but is not limited to a tris-EDTA buffer (“TE buffer”) or a phosphate buffer.

The first and second solutions may also include water, such as distilled water or deionized water.

The first and second solutions may be compatible with any appropriate analysis method or technique, though more specifically extracellular and intracellular analysis including immunofluorescent labeling and imaging; intracellular protein labeling; chromogenic staining; molecular analysis; genomic analysis or nucleic acid analysis, including, but not limited to, genomic sequencing, DNA arrays, expression arrays, protein arrays, and DNA hybridization arrays; in situ hybridization (“ISH”—a tool for analyzing DNA and/or RNA, such as gene copy number changes); polymerase chain reaction (“PCR”); reverse transcription PCR; or branched DNA (“bDNA”—a tool for analyzing DNA and/or RNA, such as mRNA expression levels) analysis. Some of the intracellular proteins which may be labeled include, but are not limited to, cytokeratin (“CK”), actin, Arp2/3, coronin, dystrophin, FtsZ, myosin, spectrin, tubulin, collagen, cathepsin D, ALDH, PBGD, Akt1, Akt2, c-myc, caspases, survivin, p27^{kip}), FOXC2, BRAF, Phospho-Akt1 and 2, Phospho-Erk1/2, Erk1/2, P38 MAPK, Vimentin, ER, PgR, PI3K, pFAK, KRAS, ALKH1, Twist1, Snail1, ZEB1, Fibronectin, Slug, Ki-67, M30, MAGEA3, phosphorylated receptor kinases, modified histones, chromatin-associated proteins, and MAGE.

The first and second solutions may be compatible with any appropriate stain, including both organic dyes and quantum dots, as acrylic media damage organic dyes due to the dehydration process and aqueous media quench quantum dots. In other words, the first and second solutions do not significantly degrade or affect any stains, whether due, at least in part, to the components of the first and second solutions or the process by which the first and second solutions are added. For example, when foregoing freezing, the stains may retain at least 75% emission after 24 hours of contact with the solutions disclosed herein or may retain at least 50% emission after 72 hours of contact with the solutions disclosed herein. The 100% emission, against which the subsequent emissions are to be compared, is the emission of the respective stains when the sample is imaged without the solutions.

### Methods

To obtain a sample, the sample may be withdrawn directly from a subject or the sample may undergo enrichment and/or isolation from a suspension. The sample may be enriched by any appropriate enrichment process including, but not limited to, density-based separation, sequential density fractionation, magnetic-activated cell sorting, fluorescence-activated cell sorting, differential lysis, depletion filters, or the like. Sequential density fractionation is a process by which a suspension is divided into fractions or a fraction of a suspension is divided into sub-fractions by a step-wise or sequential process, such that each step or sequence results in the collection or separation of a different fraction or sub-fraction from the preceding and successive steps or sequences. The sample may be obtained from other suspension components by selecting the sample with a device for picking, such as a cell picker, a pipet, a syringe, or the like.

After obtaining the sample, the sample may be placed on or in a substrate. Furthermore, the sample may be adhered to the substrate, such as with an attachment solution. For example, the sample may be re-suspended in the attachment solution in a vessel. Alternatively, the attachment solution may be added to or mixed with the sample. The re-suspended sample, which includes at least a portion of the attachment solution is dispensed onto or into the substrate by a dispenser, such as a pipet or repeating pipet, and spread across the substrate. The sample, where appropriate, may then be spread across the substrate by a spreader, such as a squeegee, a pipet tip, a blade, a two-piece spreader including a blade and a base. Alternatively, the sample may be spread across the substrate by centrifuging, wetting, or notating the substrate. The re-suspended sample is cured to adhere the re-suspended sample to the substrate. Alternatively, the re-suspended sample may be dispensed onto the substrate and cured without being spread across the substrate. Curing may occur in air, such as at room temperature; in an environmentally-controlled chamber, such as at 37° C.; or the like. Furthermore, the sample may undergo an additional fixation step, such as in formalin or any appropriate fixative, after the curing step has been completed. The sample may then undergo staining, such as within an autostainer or by manual staining.

The target analyte and non-target analyte may have a number of different types of receptor molecules located on the surface or within the analyte. Each type of receptor is a molecule capable of attaching a particular linking molecule. As a result, linking molecules can be used to classify the target analytes and/or non-target analytes and determine the specific type of target analytes and/or non-target analytes present in the suspension by conjugating linking molecules that attach to particular receptors with a particular fluorescent probe. The analytes may be classified based on various markers including nuclear markers, intracellular markers, extracellular markers (such as a membrane marker), and exclusion markers.

A stain is a compound for labeling a biological material which includes the fluorescent probe and the linking molecule. The fluorescent probe is a compound which emits light after undergoing excitation by light or electromagnetic radiation. The fluorescent probe may be used as a tracer, as a label for certain structures, as a label for biomarkers, or the like. The fluorescent probe can be distributed or can label the appropriate structure or biomarkers in manners including, but not limited to, uptake, selective uptake, diffusion, and attachment to the linking molecule. The linking molecule may be an antibody to bind to an antigen, a biomarker, a chemoattractant molecule, a protein, or the like. The linking molecule may allow for conjugation of the fluorescent probe to the target analyte, or even the non-target analyte, where appropriate. The bond, and related attraction, may be covalent or non-covalent, including bonding such as ionic, dipole-dipole interactions, London dispersion forces, van der Waal's forces, hydrophobic interactions, and hydrogen bonding. For instance, a fluorescent probe may be attached to an EpCAM antibody, thereby forming an EpCAM antibody-fluorescent probe complex. When the EpCAM antibody-fluorescent probe complex is introduced to a target material with an EpCAM biomarker, the EpCAM biomarker and the EpCAM antibody can attract and bond to each other, thereby attaching the fluorescent probe to the target material. In another example, a fluorescent probe may be attached to an avidin, such as streptavidin or neutravidin, thereby forming an avidin-fluorescent probe complex. When the avidin-fluorescent probe complex is introduced to a target material with a biotin molecule on the outer surface, the biotin and avidin attract and bond to each other, thereby attaching the fluorescent probe to the target material.

A set of stains, which includes at least two stains and may include up to 4, 6, 8, 10, or more, may be used to multiplex the target material, such that each stain binds to a different antigen on or within the target material. For example, each type of fluorescent probe emits light in a narrow wavelength range of the electromagnetic spectrum when an appropriate stimulus, such as light with a shorter wavelength, is applied. A first fluorescent probe that emits light having a wavelength in the green channel can be attached to a first linking molecule that binds specifically to a first type of receptor, while a second fluorescent probe that emits light having a wavelength in the red channel can be attached to a second linking molecule that binds specifically to a second type of receptor. The color observed, based on the wavelength emitted, as a result of stimulating the respective fluorescent probe identifies the type of receptor, and because receptors can be unique to particular target analytes and/or non-target analytes, the color can also be used to identify the target particle. This allows for detection of multiple receptors on the target material at any given time.

The fluorescent probe may be a reactive dye, an organic dye, a fluorescent protein, a quantum dot (including QD500, QD525, QD625, and QD800), non-protein organic molecules, or the like. Fluorescent probes may include, but are not limited to 1,5 IAEDANS; 1,8-ANS; 4-Methylumbelliferone; 5-carboxy-2,7-dichlorofluorescein; 5-Carboxyfluorescein (5-FAM); 5-Carboxynapthofluorescein; 5-Carboxytetramethylrhodamine (5-TAMRA); 5-FAM (5-Carboxyfluorescein); 5-HAT (Hydroxy Tryptamine); 5-Hydroxy Tryptamine (HAT); 5-ROX (carboxy-X-rhodamine); ((5-TAMRA (5-Carboxytetramethylrhodamine); 6-Carboxyrhodamine 6G; 6-CR 6G; 6-JOE; 7-Amino-4-methylcoumarin; 7-Aminoactinomycin D (7-AAD); 7-Hydroxy-4-methylcoumarin; 9-Amino-6-chloro-2-methoxyacridine; ABQ; Acid Fuchsin; ACMA (9-Amino-6-chloro-2-methoxyacridine); Acridine Orange; Acridine Red; Acridine Yellow; Acriflavin; Acriflavin Feulgen SITSA; Aequorin (Photoprotein); AutoFluorescent Protein; Alexa Fluor 350™; Alexa Fluor 430™; Alexa Fluor 488™; Alexa Fluor 532™; Alexa Fluor 546™; Alexa Fluor 568™; Alexa Fluor 594™; Alexa Fluor 633™; Alexa Fluor 647™; Alexa Fluor 660™; Alexa Fluor 680™; Alizarin Complexon; Alizarin Red; Allophycocyanin (APC); AMC; AMCA-S; AMCA (Aminomethylcoumarin); AMCA-X; Amino actinomycin D; Aminocoumarin; Aminomethylcoumarin (AMCA); Anilin Blue; Anthrocyl stearate; APC (Allophycocyanin); APC-Cy7; APTRA-BTC; APTS; Astrazon Brilliant Red 4G; Astrazon Orange R; Astrazon Red 6B; Astrazon Yellow 7 GLL; Atabrine; ATTO-TAG™ CBQCA; ATTO-TAG™ FQ; Auramine; Aurophosphine G; Aurophosphine; BAO 9(Bisaminophenyloxadiazole); BCECF (high pH); BCECF (low pH); Berberine Sulphate; Beta Lactamase; BFP blue shifted GFP (Y66H; Blue Fluorescent Protein); BFP/GFP FRET; Bimane; Bisbenzamide; Bisbenzimide (Hoechst); bis-BTC; Blancophor FFG; Blancophor SV; BOBO™-1; BOBO™-3; Bodipy 492/515; Bodipy 493/503; Bodipy 500/510; Bodipy 505/515; Bodipy 530/550; Bodipy 542/563; Bodipy 558/568; Bodipy 564/570; Bodipy 576/589; Bodipy 581/591; Bodipy 630/650-X; Bodipy 650/665-X; Bodipy 665/676; Bodipy Fl; Bodipy FL ATP; Bodipy Fl-Ceramide; Bodipy R6G SE; Bodipy TMR; Bodipy TMR-X conjugate; Bodipy TMR-X, SE; Bodipy TR; Bodipy TR ATP; Bodipy TR-X SE; BO-PRO™-1; BO-PRO™-3; Brilliant Sulphoflavin FF; Brilliant Violet 421; Brilliant Violet 510; Brilliant Violet 605; Brilliant Violet 650; Brilliant Violet 711; Brilliant Violet 786; BTC; BTC-5N; Calcein; Calcein Blue; Calcium Crimson™; Calcium Green; Calcium Green-1; Calcium Green-2; Calcium Green-5N; Calcium Green-C18; Calcium Orange; Calcofluor White; Carboxy-X-hodamine (5-ROX); Cascade Blue™; Cascade Yellow; Catecholamine; CCF2 (GeneBlazer); CFDA; CFP (Cyan Fluorescent Protein); FP/YFP FRET; Chlorophyll; Chromomycin A; Chromomycin A; CL-NERF; CMFDA; Coelenterazine; Coelenterazine cp; Coelenterazine f; Coelenterazine fcp; Coelenterazine h; Coelenterazine hcp; Coelenterazine ip; Coelenterazine n; Coelenterazine O; Coumarin Phalloidin; C-phycocyanine; CPM Methylcoumarin; CTC; CTC Formazan; Cy2™; Cy3.1 8; Cy3.5™; Cy3™; Cy5.1 8; Cy5.5™; Cy5™; Cy7™; Cyan GFP; cyclic AMP Fluorosensor (FiCRhR); CyQuant Cell Proliferation Assay; Dabcyl; Dansyl; Dansyl Amine; Dansyl Cadaverine; Dansyl Chloride; Dansyl DHPE; DAPI; Dapoxyl; Dapoxyl 2; Dapoxyl 3; DCFDA; DCFH (Dichlorodihydrofluorescein Diacetate); DDAO; DHR (Dihydorhodamine 123); Di-4-ANEPPS; Di-8-ANEPPS; DiA (4-Di-16-ASP); Dichlorodihydrofluorescein Diacetate (DCFH); DiD-Lipophilic Tracer; DiD (DiIC18(5)); DIDS; Dihydorhodamine 123 (DHR); DiI (DiIC18(3)); Dinitrophenol; DiO (DiOC18(3)); DiR; DiR (DiIC18(7)); DM-NERF (high pH); DNP; Dopamine; DsRed; DTAF; DY-630-NHS; DY-635-NHS; EBFP (Enhanced Blue Fluorescent Protein); ECFP (Enhanced Cyan Fluorescent Protein); EGFP (Enhanced Green Fluorescent Protein); ELF 97; Eosin; ER-Tracker™ Green; ER-Tracker™ Red; ER-Tracker™ Blue-White DPX; Erythrosin; Erythrosin ITC; Ethidium Bromide; Ethidium homodimer-1 (EthD-1); Euchrysin; EukoLight; Europium (III) chloride; EYFP (Enhanced Yellow Fluorescent Protein); Fast Blue; FDA; FIF (Formaldehyde Induced Fluorescence); FITC; FITC Antibody; Flazo Orange; Fluo-3; Fluo-4; Fluorescein (FITC); Fluorescein Diacetate; Fluoro-Emerald; Fluoro-Gold (Hydroxystilbamidine); Fluor-Ruby; FluorX; FM 1-43™; FM 4-46; Fura Red™ (high pH); Fura Red™/Fluo-3; Fura-2, high calcium; Fura-2, low calcium; Fura-2/BCECF; Genacryl Brilliant Red B; Genacryl Brilliant Yellow 10GF; Genacryl Pink 3G; Genacryl Yellow 5GF; GeneBlazer (CCF2); GFP (S65T); GFP red shifted (rsGFP); GFP wild type, non-UV excitation (wtGFP); GFP wild type, UV excitation (wtGFP); GFPuv; Gloxalic Acid; Granular Blue; Haematoporphyrin; Hoechst 33258; Hoechst 33342; Hoechst 34580; HPTS; Hydroxycoumarin; Hydroxystilbamidine (FluoroGold); Hydroxytryptamine; Indo-1, high calcium; Indo-1, low calcium; Indodicarbocyanine (DiD); Indotricarbocyanine (DiR); Intrawhite Cf JC-1; JO-JO-1; JO-PRO-1; LaserPro; Laurodan; LDS 751; Leucophor PAF; Leucophor SF; Leucophor WS; Lissamine Rhodamine; Lissamine Rhodamine B; Calcein/Ethidium homodimer; LOLO-1; LO-PRO-1; Lucifer Yellow; Lyso Tracker Blue; Lyso Tracker Blue-White; Lyso Tracker Green; Lyso Tracker Red; Lyso Tracker Yellow; LysoSensor Blue; LysoSensor Green; LysoSensor Yellow/Blue; Mag Green; Magdala Red (Phloxin B); Mag-Fura Red; Mag-Fura-2; Mag-Fura-5; Mag-Indo-1; Magnesium Green; Magnesium Orange; Malachite Green; Marina Blue; Maxilon Brilliant Flavin 10 GFF; Maxilon Brilliant Flavin 8 GFF; Merocyanin; Methoxycoumarin; Mitotracker Green; Mitotracker Orange; Mitotracker Red; Mitramycin; Monobromobimane; Monobromobimane (mBBr-GSH); Monochlorobimane; MPS (Methyl Green Pyronine Stilbene); mStrawberry; NBD; NBD Amine; Nile Red; Nitrobenzoxadidole; Noradrenaline; Nuclear Fast Red; Nuclear Yellow; Nylosan Brilliant Iavin E8G; Oregon Green™; Oregon Green™ 488; Oregon Green™ 500; Oregon Green™ 514; Pacific Blue; Pararosaniline (Feulgen); PBFI; PE-Cy5; PE-Cy7; PerCP; PerCP-Cy5.5; PE-TexasRed (Red 613); Phloxin B (Magdala Red); Phorwite AR; Phorwite BKL; Phorwite Rev; Phorwite RPA; Phosphine 3R; PhotoResist; Phycoerythrin B; Phycoerythrin R; PKH26 (Sigma); PKH67; PMIA; Pontochrome Blue Black; POPO-1; POPO-3; PO-PRO-1; PO-PRO-3; Primuline; Procion Yellow; Propidium Iodid (PI); Pyrene; Pyronine; Pyronine B; Pyrozal Brilliant Flavin 7GF; QSY 7; Quinacrine Mustard; Red 613 (PE-TexasRed); Resorufin; RFP; RH 414; Rhod-2; Rhodamine; Rhodamine 110; Rhodamine 123; Rhodamine 5 GLD; Rhodamine 6G; Rhodamine B; Rhodamine B 200; Rhodamine B extra; Rhodamine BB; Rhodamine BG; Rhodamine Green; Rhodamine Phallicidine; Rhodamine Phalloidine; Rhodamine Red; Rhodamine WT; Rose Bengal; R-phycocyanine; R-phycoerythrin; rsGFP (red shifted GFP (S65T)); S65A; 565C; 865L; S65T; Sapphire GFP; SBFI; Serotonin; Sevron Brilliant Red 2B; Sevron Brilliant Red 4G; Sevron Brilliant Red B; Sevron Orange; Sevron Yellow L; sgGFP™ (super glow GFP; SITS (Primuline); SITS (Stilbene Isothiosulphonic Acid); SNAFL calcein; SNAFL-1; SNAFL-2; SNARF calcein; SNARF1; Sodium Green; SpectrumAqua; SpectrumGreen; SpectrumOrange; Spectrum Red; SPQ (6-methoxy-N-(3-sulfopropyl)quinolinium); Stilbene; Sulphorhodamine B can C; Sulphorhodamine G Extra; SYTO 11; SYTO 12; SYTO 13; SYTO 14; SYTO 15; SYTO 16; SYTO 17; SYTO 18; SYTO 20; SYTO 21; SYTO 22; SYTO 23; SYTO 24; SYTO 25; SYTO 40; SYTO 41; SYTO 42; SYTO 43; SYTO 44; SYTO 45; SYTO 59; SYTO 60; SYTO 61; SYTO 62; SYTO 63; SYTO 64; SYTO 80; SYTO 81; SYTO 82; SYTO 83; SYTO 84; SYTO 85; SYTOX Blue; SYTOX Green; SYTOX Orange; SYTOX Red; Tetracycline; Tetramethylrhodamine (TRITC); Texas Red™; Texas Red-X™ conjugate; Thiadicarbocyanine (DiSC3); Thiazine Red R; Thiazole Orange; Thioflavin 5; Thioflavin S; Thioflavin TCN; Thiolyte; Thiozole Orange; Tinopol CBS (Calcofluor White); TMR; TO-PRO-1; TO-PRO-3; TO-PRO-5; TOTO-1; TOTO-3; TriColor (PE-Cy5); TetramethylRodamineIsoThioCyanate; True Blue; TruRed; Tubulin Tracker™ Green; Ultralite; Uranin B; Uvitex SFC; wt GFP (wild type GFP); WW 781; X-Rhodamine; XRITC; Xylene Orange; Y66F; Y66H; Y66W; Yellow GFP (Yellow shifted); Green Fluorescent Protein; YFP (Yellow Fluorescent Protein); YO-PRO-1; YO-PRO-3; YOYO-1; YOYO-3; and, combinations and derivatives thereof.

After staining, the first or second solution may then be added to the substrate and the associated sample. To do so, the solution may be added directly on top of the sample and may be spread across, or the solution may be added to the sample followed by a coverslip, such that covering the sample with the coverslip draws the solution across the sample, such as by capillary action. After curing, such as for 5 minutes to 24 hours, the substrate, including the sample and the first or second solution, may be frozen to −80° C., including to −20° C., or may be imaged. Furthermore, a target analyte of the sample may be picked through the first or second solution, such as by a picking device, including a pipet tip, a hydraulic picker, or mechanical picker.

The substrate may be a microscope slide, a positively charged microscope slide, a coated microscope slide, a porous slide, a micro-well slide, a well plate, a coverslip, a cell microarray, or the like. The substrate may be any appropriate material, including, but not limited to, glass, plastic, ceramic, metal, or the like.

The foregoing description, for purposes of explanation, used specific nomenclature to provide a thorough understanding of the disclosure. However, it will be apparent to one skilled in the art that the specific details are not required in order to practice the systems and methods described herein. The foregoing descriptions of specific embodiments are presented by way of examples for purposes of illustration and description. They are not intended to be exhaustive of or to limit this disclosure to the precise forms described. Many modifications and variations are possible in view of the above teachings. The embodiments are shown and described in order to best explain the principles of this disclosure and practical applications, to thereby enable others skilled in the art to best utilize this disclosure and various embodiments with various modifications as are suited to the particular use contemplated. It is intended that the scope of this disclosure be defined by the following claims and their equivalents:

I/We claim:

1. A solution comprising:
a polyol having a concentration of 5-30% by mass;
polyvinyl alcohol (“PVA”) having a concentration of 5-20% by mass; and
a buffer.

2. The solution of claim 1, wherein the polyol is glycerol, propylene glycol, or pentaerythritol.

3. The solution of claim 2, wherein the buffer is tris-EDTA buffer (“TE buffer”) or a phosphate buffer.

4. The solution of claim 1, wherein the buffer is TE buffer or a phosphate buffer.

5. The solution of claim 1, wherein the polyol is glycerol and has a concentration of 10% by mass, wherein the PVA has a concentration of 10% by mass, wherein the buffer is TE buffer and the tris has a concentration of 20 mM and the EDTA has a concentration of 2 mM.

6. A system comprising:
a solution comprising
a polyol having a concentration of 5-30% by mass,
polyvinyl alcohol (“PVA”) having a concentration of 5-20% by mass, and a buffer; and
a set of stains comprising
at least one quantum dot, and
at least one fluorophore.

7. The system of claim 6, wherein the polyol is glycerol, propylene glycol, or pentaerythritol.

8. The system of claim 7, wherein the buffer is tris-EDTA buffer (“TE buffer”) or a phosphate buffer.

9. The system of claim 6, wherein the buffer is TE buffer or a phosphate buffer.

10. The system of claim 6, wherein the polyol is glycerol and has a concentration of 10% by mass, wherein the PVA has a concentration of 10% by mass, wherein the buffer is TE buffer and the tris has a concentration of 20 mM and the EDTA has a concentration of 2 mM.

11. A method comprising:
providing a sample comprising a target material;
labeling the target material with a set of stains comprising
at least one quantum dot, and
at least one fluorophore; and
covering the sample with a solution comprising
a polyol having a concentration of 5-30% by mass,
polyvinyl alcohol (“PVA”) having a concentration of 5-20% by mass, and a buffer,
wherein the covering step occurs after the labeling step.

12. The method of claim 11, further comprising the step of:
imaging the sample,
wherein the imaging step occurs after the covering step.

13. The method of claim 12, wherein the set of stains retains at least 75% emission after 24 hours of contact with the solution, and wherein 100% emission is the emission of the set of stains when the sample is imaged without the solution.

14. The method of claim 13, wherein the polyol is glycerol, propylene glycol, or pentaerythritol.

15. The method of claim 14, wherein the buffer is tris-EDTA buffer (“TE buffer”) or a phosphate buffer.

16. The method of claim 12, wherein the set of stains retains at least 50% emission after 72 hours of contact with the solution, and wherein 100% emission is the emission of the set of stains when the sample is imaged without the solution.

17. The method of claim 16, wherein the polyol is glycerol, propylene glycol, or pentaerythritol.

18. The method of claim 17, wherein the buffer is tris-EDTA buffer (“TE buffer”) or a phosphate buffer.

19. The method of claim 11, wherein the buffer is TE buffer or a phosphate buffer.

20. The method of claim 11, wherein the polyol is glycerol and has a concentration of 10% by mass, wherein the PVA has a concentration of 10% by mass, wherein the buffer is TE buffer and the tris has a concentration of 20 mM and the EDTA has a concentration of 2 mM.
